(19) United States
(12) Patent Application Publication
(10) Pub. No.: US 2025/0353885 A1
(43) Pub. Date: Nov. 20, 2025

(54) Use of plant peptides against metabolic abnormalities

(71) Applicant: Greenyn Biotechnology Co., Ltd, Taichung City (TW)

(72) Inventors: Pang-Kuei Hsu, Taichung City (TW); Yu Cheng Lin, Taichung City (TW); Chia-Feng Wu, Taichung City (TW)

(21) Appl. No.: 18/667,547

(22) Filed: May 17, 2024

(51) Int. Cl. C07K 14/415; A61K 38/00; A61P 3/00; C07K 1/14; C07K 1/34

(52) U.S. Cl. CPC C07K 14/415; A61P 3/00; C07K 1/145; C07K 1/34; A61K 38/00

(57) ABSTRACT

A use of a plant peptide group in anti-metabolic abnormalities is provided. More specifically, the plant peptide group disclosed herein has the activity to regulate blood glucose, blood lipids, and blood pressure at the same time. Therefore, administering an effective amount of the plant peptide group to an individual can effectively produce the effect of preventing or improving such metabolic diseases as hypertension, hyperlipidemia, and hyperglycemia. The plant peptide group is obtained by separation from an initial product of a plant-based raw material.

### BACKGROUND OF THE INVENTION

### 1. Technical Field

The present invention relates to a use of a group of peptides. More particularly, the invention relates to a use of a plant peptide group in anti-metabolic abnormalities.

### 2. Description of Related Art

People nowadays have a higher level of health awareness than before. Therefore, instead of or in addition to vitamins, minerals, and other basic nutritional supplements, consumers tend to buy nutritional supplements with special functions to meet their needs. With the advancement of biotechnology, many manufacturers can now use special processes such as fermentation and hydrolysis to derive active ingredients with specific functions from food, and these ingredients can be used as the active ingredients of nutritional supplements to enhance the effectiveness of health promotion.

According to statistics, more than one fourth of the people in Taiwan have such metabolic diseases as hypertension, hyperglycemia, and hyperlipidemia. Apart from heredity, the causes of such metabolic diseases are closely related to one's diet and way of living. Some examples of the causes of metabolic diseases are an abnormal daily routine, high stress in life, a lack of physical exercise, and a diet dominated by deep-fried food. While metabolic diseases do not seem to have a significant impact on health in the short run, serious diseases such as stroke, heart attack, and diabetes may result eventually. Given the relatively great difficulty of changing one's diet and way of living, most people choose to regulate their blood pressure, blood glucose, and blood lipids by taking nutritional supplement. Currently, however, the market still lacks a nutritional supplement that can improve blood pressure, blood glucose, and blood lipids at the same time.

### BRIEF SUMMARY OF THE INVENTION

The primary objective of the present invention is to provide a use of a plant peptide group in anti-metabolic abnormalities. The plant peptide group includes a plurality of small-molecule active peptides that work synergistically to regulate blood glucose, blood lipids, and blood pressure at the same time. Therefore, administering the plant peptide group disclosed herein is effective in simultaneously improving or treating diseases associated with hypertension, hyperglycemia, and hyperlipidemia.

To achieve the above objective, the present invention discloses a use of a plant peptide group in anti-metabolic abnormalities. The plant peptide group is separated from an initial product of a plant-based raw material and includes at least six small-molecule peptides that can work synergistically to control the mitogen-activated protein kinase (MAPK) signal pathway and the insulin receptor substrate (IRS)/phosphorylated insulin receptor (p-IR)/phosphoinositide 3-kinase (PI3K)/protein kinase B (Akt) signal pathways in an individual, thereby simultaneously improving or treating multiple metabolic diseases associated with an imbalance of such metabolic indicators as blood pressure, blood lipids, and blood glucose.

The initial product of the plant-based raw material is obtained by performing a two-stage extraction process on the plant-based raw material. The first stage of the extraction process is water-based extraction, and the second stage of the extraction process is supercritical extraction.

Administering an effective amount of the plant peptide group disclosed herein or of a composition containing the plant peptide group to an individual can produce the effect of combating or improving multiple metabolic diseases at the same time, in particular of simultaneously treating or preventing diseases associated with hypertension, hyperglycemia, and hyperlipidemia. For humans, the effective dosage of the plant peptide group is 5 mg/kg/day.

In one embodiment of the present invention, the plant peptide group includes peptides corresponding to sections (L1) to (L6) of a chromatogram of high-performance liquid chromatography (HPLC), wherein sections (L1) to (L6) correspond to molecular weights in the range from 300 Da to 3000 Da, with the retention time of section (L1) being 10.5-11.5 minutes, the retention time of section (L2) being 12.5-13.5 minutes, the retention time of section (L3) being 14.5-15.5 minutes, the retention time of section (L4) being 16.5-17.5 minutes, the retention time of section (L5) being 17.5-18.5 minutes, and the retention time of section (L6) being 24.5-25.5 minutes.

The HPLC chromatogram is obtained by subjecting the initial product of the plant-based raw material to a pre-treatment process and then performing an analysis with an HPLC instrument under predetermined analysis conditions, wherein the analysis conditions are as follows: the detection wavelength is 220 nm, the separation column is an octadecyl carbon chain (C18)-bonded silica column (RP-C18), the column temperature is 60° C., eluent A is 5% acetonitrile containing 0.10% trifluoroacetic acid, eluent B is 95% acetonitrile containing 0.085% trifluoroacetic acid, and the mobile phase includes 10%-20% eluent B used from the 0-minute time point through the 10-minute time point, 20%-30% eluent B used from the 10-minute time point through the 25-minute time point, and 30%-60% eluent B used from the 20-minute time point through the 60-minute time point.

In another embodiment of the present invention, the pre-treatment process includes: dissolving the initial product of the plant-based raw material in 30% acetonitrile, and then performing ultrasonic extraction to obtain a supernatant; and filtering the supernatant to obtain a filtrate to be analyzed by HPLC.

In the foregoing pre-treatment process, the filtering is carried out using a filter whose pore size is in the range from 0.20 to 0.25 μm, and the filter may be a membrane filter or a mesh sieve.

In another embodiment of the present invention, the peptides corresponding to sections (L1) to (L6) and included in the plant peptide group capable of anti-metabolic abnormalities have molecular weights in the range from 700 Da to 2400 Da.

### BRIEF DESCRIPTION OF THE SEVERAL VIEWS OF THE DRAWINGS

FIG. 1 shows chromatographic analysis result (1) of a peptide group derived from a product of extraction of bitter melon 1.

FIG. 2 shows chromatographic analysis result (2) of a peptide group derived from a product of extraction of watermelon 1.

FIG. 3 shows chromatographic analysis result (3) of a peptide group derived from a product of extraction of watermelon 2.

FIG. 4 shows chromatographic analysis result (4) of a peptide group derived from a product of extraction of pumpkin 1.

FIG. 5 shows chromatographic analysis result (5) of a peptide group derived from a product of extraction of pumpkin 2.

FIG. 6 shows chromatographic analysis result (6) of a peptide group derived from a product of extraction of bitter melon 2.

FIG. 7 shows chromatographic analysis result (7) of a peptide group derived from a product of extraction of wild bitter melon 1.

FIG. 8 shows chromatographic analysis result (8) of a peptide group derived from a product of extraction of wild bitter melon 2.

FIG. 9 shows chromatographic analysis result (9) of a peptide group derived from a product of extraction of bitter melon 3.

FIG. 10 shows chromatographic analysis result (10) of a peptide group derived from a product of extraction of bitter melon 4.

FIG. 11 shows chromatographic analysis result (11) of a peptide group derived from a product of extraction of bitter melon 5.

FIG. 12 shows a comparison between the chromatogram in FIG. 1 and the chromatograms of comparative samples 1-7.

FIG. 13 shows the analysis result of the fasting blood glucose value of each group of rats in animal test (1) at the end of week 8 of the test.

FIG. 14 shows the analysis result of the glycated hemoglobin in blood of each group of rats in animal test (1) at the end of week 8 of the test.

FIG. 15 shows the analysis result of the fasting blood glucose value of each group of rats in animal test (2) at the end of week 8 of the test.

FIG. 16 shows the analysis result of the body weight of each group of rats in animal test (3) at the end of week 8 of the test.

FIG. 17 shows the analysis result of the body fat of each group of rats in animal test (3) at the end of week 8 of the test.

FIG. 18 shows the analysis result of the triglycerides in blood of each group of rats in animal test (3) at the end of week 8 of the test.

FIG. 19 shows the analysis result of the total cholesterol in blood of each group of rats in animal test (3) at the end of week 8 of the test.

FIG. 20 shows the analysis result of the low-density cholesterol in blood of each group of rats in animal test (3) at the end of week 8 of the test.

FIG. 21 shows the analysis result of the changes in after-meal blood glucose of each group of test subjects.

FIG. 22 shows the analysis result of the area under the after-meal blood glucose curve of each group of test subjects within a period of two hours.

### DETAILED DESCRIPTION OF THE INVENTION

The present invention discloses a use of a plant peptide group in anti-metabolic abnormalities. More specifically, the plant peptide group disclosed herein is capable of reducing blood glucose, blood pressure, blood lipids, and lipogenesis; therefore, administering the plant peptide group disclosed herein or a composition containing the same to an individual can improve such indicators of the individual as blood pressure, blood glucose, blood lipids, and body weight by gradually bringing those indicators back to their respective normal ranges, thereby producing the effect of treating or improving metabolic diseases such as diabetes, kidney disease, hypertension, and hyperlipidemia, and of lowering the risk of having complications or symptoms related to the aforesaid diseases.

In one embodiment of the present invention, the plant peptide group disclosed herein is obtained by separation from an initial product of a plant-based raw material, and the plant peptide group is composed of peptides corresponding to sections (L1) to (L6) of an HPLC chromatogram, wherein sections (L1) to (L6) correspond to molecular weights ranging from 300 Da to 3000 Da and have retention times ranging from 10.5 to 25.5 minutes.

The HPLC chromatogram is obtained by performing a pre-treatment process on the initial product of the plant-based raw material and then performing an HPLC analysis under predetermined analysis conditions.

More specifically, the plant peptide group disclosed herein is separated from the initial product of the plant-based raw material, and the initial product of the plant-based raw material is obtained by performing water-based extraction and supercritical extraction on the plant-based raw material. The plant-based raw material has at least one of the following features: being able to inhibit dipeptidyl peptidase-4, being able to inhibit α-amylase, being able to inhibit glucose transporter 2 (GLUT2), being able to inhibit sodium/glucose cotransporter 1 (SGLT1), being able to inhibit α-glucosidase, and being able to regulate insulin.

The plant-based raw material is a cucurbitaceous plant such as watermelon, pumpkin, bitter melon, or calabash.

In the present invention, “the initial product of the plant-based raw material” is a product obtained by performing a combination of water-based extraction and supercritical extraction on the plant-based raw material and then removing the scum and/or residue, and the product contains the plant peptide group disclosed herein.

In the present invention, the “plant-based raw material” may be a plant of such a classification as Amaranthus sp., Phaseolus sp., Glycine sp., Avena sp., Cucurbitaceae, Cuminum, Chenopodium sp., Orus, Juglans sp., Hordeum sp., Spinacia sp., Vugna sp., Triticum sp., Linum sp., Phalaris sp., Moringa sp., Theobroma sp., or Zea sp., such as bitter melon (Momordica charantia), wild bitter melon, cucumber (Cucumis sativus), pumpkin (Cucurbita pepo), calabash (Lagenaria siceraria), watermelon (Citrullus lanatus), ginger root, gualou (Trichosanthes kirilowii), Zinia elegans, Medicago truncatula, grape (Vitis), grapefruit (Citrus x paradisi), Sambucus nigra, Arabidopsisthali, or rice (e.g., Oryza sativa).

As used herein, the term “composition” refers to a substance containing the plant peptide group disclosed herein in a concentration of 0.001%-100%. If not composed entirely of the plant peptide group disclosed herein, such a composition further includes a vehicle and/or excipient that is pharmaceutically acceptable or is acceptable in food. Such a composition may be prepared in different dosage forms as appropriate, such as tablets, powder, and aqueous solutions, and may be used in different dosages, depending on such parameters as the administration requirement, the individuals to whom the composition is administered, and the number of times of administration. For example, such a composition may be a nutritional supplement, a food, or a drug.

As used herein, the term “effective amount” or “effective dosage” refers to the minimum dosage at which the plant peptide group disclosed herein should be administered to an individual in order to be active in the individual's body.

The plant peptide group disclosed herein can be separated from different kinds of plant-based raw materials consistently, and can reduce blood glucose, body weight, body fat, triglycerides in blood, total cholesterol in blood, low-density fat in blood, and blood pressure simultaneously. In other words, administering a predetermined amount of the plant peptide group disclosed herein or of a composition containing the plant peptide group can effectively and simultaneously delay or improve diseases separately attributable to blood lipids, blood pressure, and abnormal metabolism of blood glucose. The present invention makes it possible to prevent or improve multiple metabolic diseases at the same time, thereby reducing not only the discomfort caused to consumers by taking several different nutritional supplements, but also the risk of interaction between nutritional supplements and/or drugs.

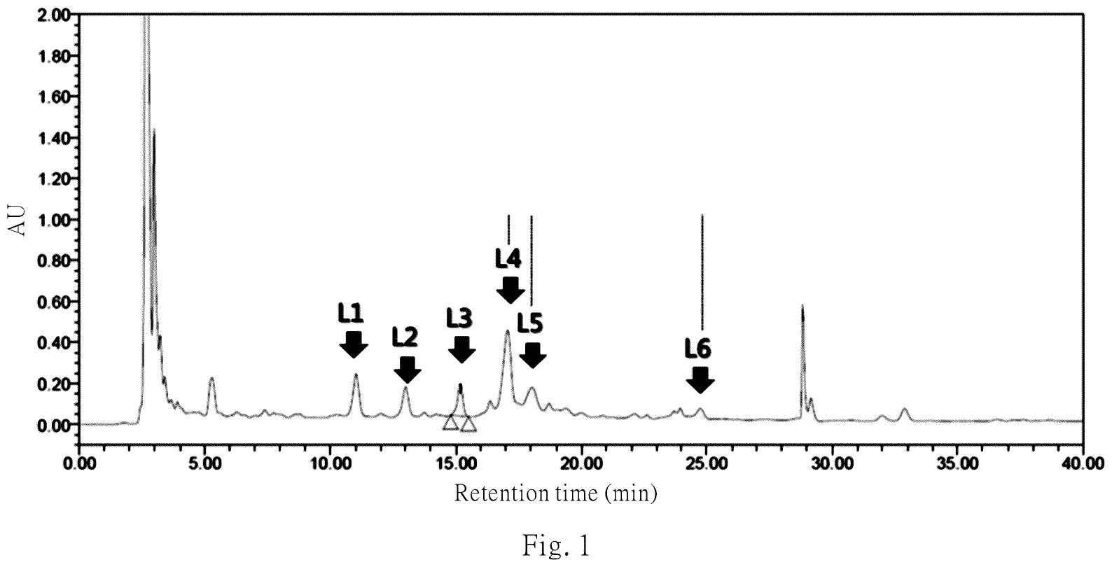

The plant peptide group disclosed in the first embodiment of the present invention includes a group of peptides, or more particularly a plurality of small-molecule functional peptides isolated from an initial product of a plant-based raw material, wherein the initial product of the plant-based raw material was obtained by sequentially performing a water-based extraction process and a supercritical extraction process on bitter melon. More specifically, the plant peptide group includes peptides corresponding to sections (L1) to (L6) of the chromatogram in FIG. 1, wherein sections (L1) to (L6) have retention times ranging from 10.5 to 25.5 minutes and correspond to molecular weights ranging from 300 Da to 3000 Da.

Sections (L1) to (L6) were obtained by performing a pre-treatment process on the initial product of the plant-based raw material and then performing an HPLC analysis under predetermined analysis conditions. The analysis conditions include: a detection wavelength of 220 nm; an octadecyl carbon chain (C18)-bonded silica column (RP-C18) serving as the separation column; a column temperature of 60° C.; eluent A, which was 5% acetonitrile containing 0.10% trifluoroacetic acid; eluent B, which was 95% acetonitrile containing 0.085% trifluoroacetic acid; and a mobile phase including 10%-20% eluent B used from the 0-minute time point through the 10-minute time point, 20%-30% eluent B used from the 10-minute time point through the 25-minute time point, and 30%-60% eluent B used from the 20-minute time point through the 60-minute time point.

The retention time of section (L1) was 10.5-11.5 minutes, the retention time of section (L2) was 12.5-13.5 minutes, the retention time of section (L3) was 14.5-15.5 minutes, the retention time of section (L4) was 16.5-17.5 minutes, the retention time of section (L5) was 17.5-18.5 minutes, and the retention time of section (L6) was 24.5-25.5 minutes.

### the Pre-Treatment Process Included the Following Steps:

Step a: dissolving the initial product of the plant-based raw material in 30% acetonitrile, and then performing ultrasonic extraction to obtain a supernatant; and
Step b: filtering the supernatant to obtain a filtrate to be analyzed by HPLC.

Sections (L1) to (L6) of the chromatogram in FIG. 1 were obtained by a gel permeation chromatography (GPC) analysis and were compared with the GPC chromatograms of the standards, and it was estimated and subsequently confirmed that the molecular weights corresponding to sections (L1) to (L6) were in the range from 300 to 3000 Da. The standards were cytidine (whose molecular weight is 243 Da), vitamin B12 (whose molecular weight is 1350 Da), aprotinin (whose molecular weight is 6500 Da), cytochrome (whose molecular weight is 12000 Da), and myoglobin (whose molecular weight is 17600 Da), and peaks corresponding to the aforesaid substances sequentially occurred in a descending order of molecular weight, namely in the order from myoglobin (10.226 min) to cytochrome (10.658 min), aprotinin (11.864 min), vitamin B12 (13.787 min), and cytidine (14.835 min).

In addition, the plant peptide group went through a simulated digestion test before another HPLC analysis was performed. The difference in area (expressed in percentage) of each of the foregoing chromatogram sections after the test is shown in Table 1 below.

TABLE 1
Analysis results of sections (L1) to (L6) in FIG. 1

| | | | Difference in the area |
|---|---|---|---|
| | | | under the curve of each |
| | Retention | Proportion | section after simulated |
| Section | time | (%) | digestion (%) |
| L1 | 10.872 | 78.65 | −41.52 |
| L2 | 12.81 | 89.87 | −42.93 |
| L3 | 15.004 | 94.05 | +14.57 |
| L4 | 16.858 | 87.20 | +58.57 |
| L5 | 17.802 | 87.36 | +166.01 |
| L6 | 24.515 | 93.56 | −22.51 |

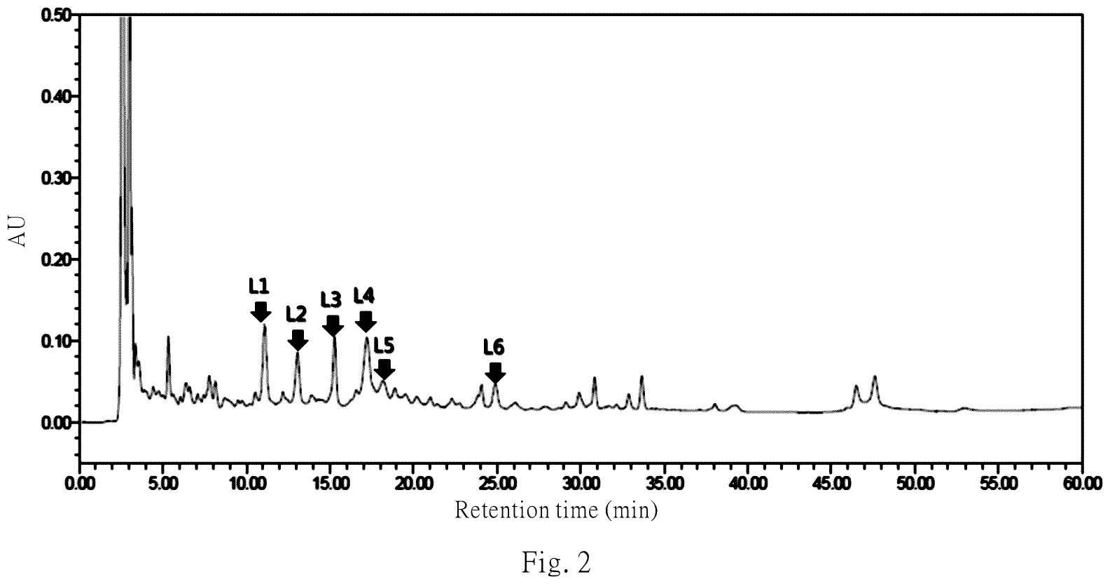

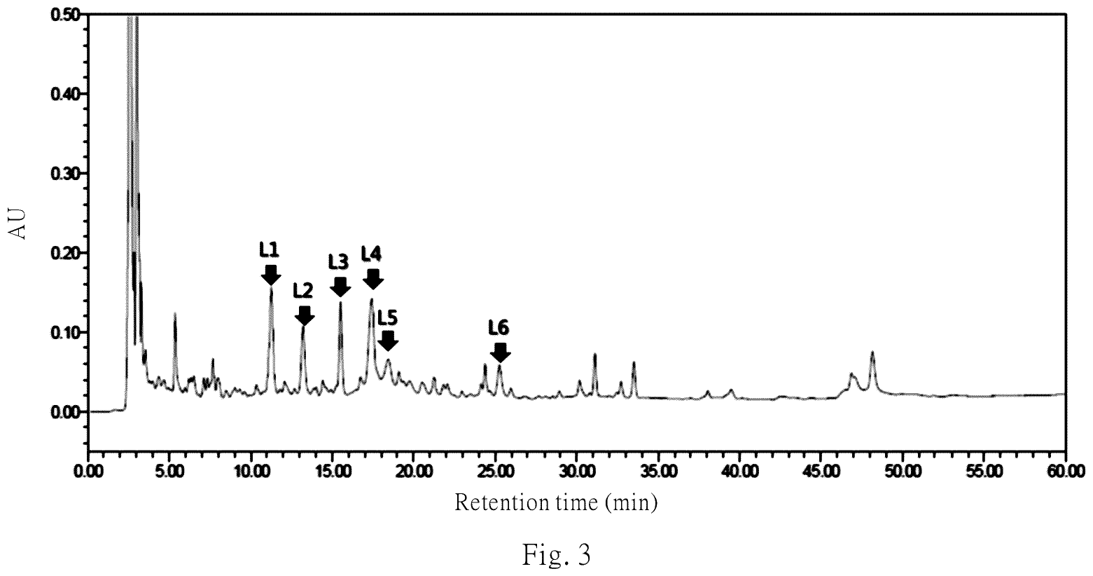

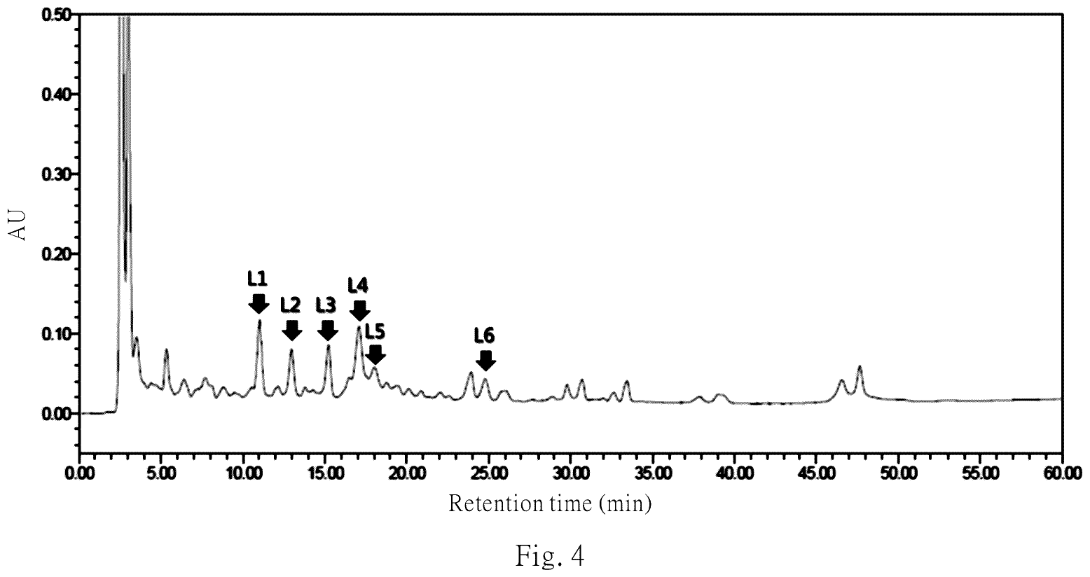

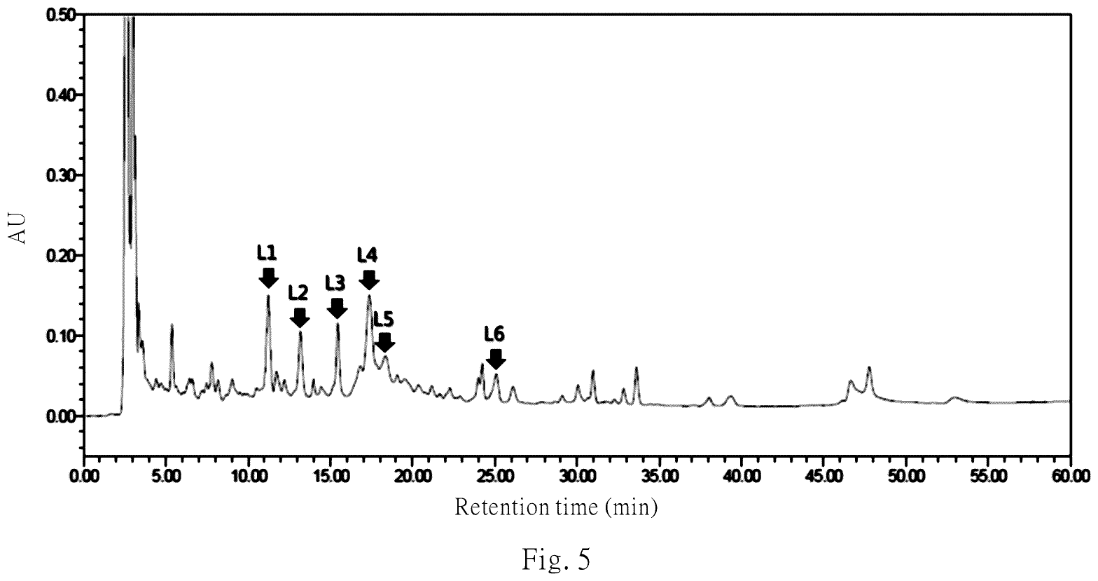

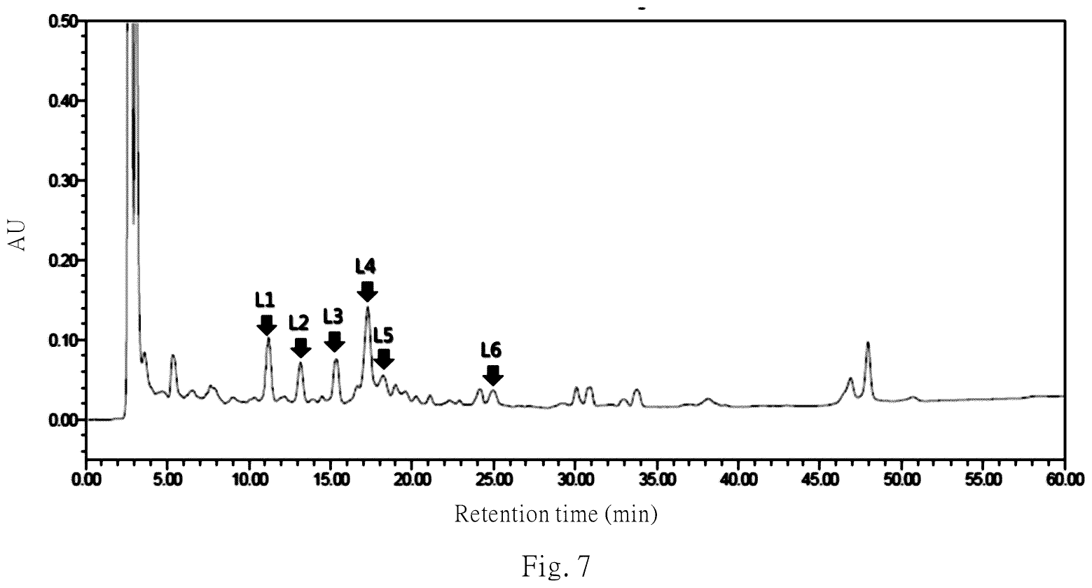

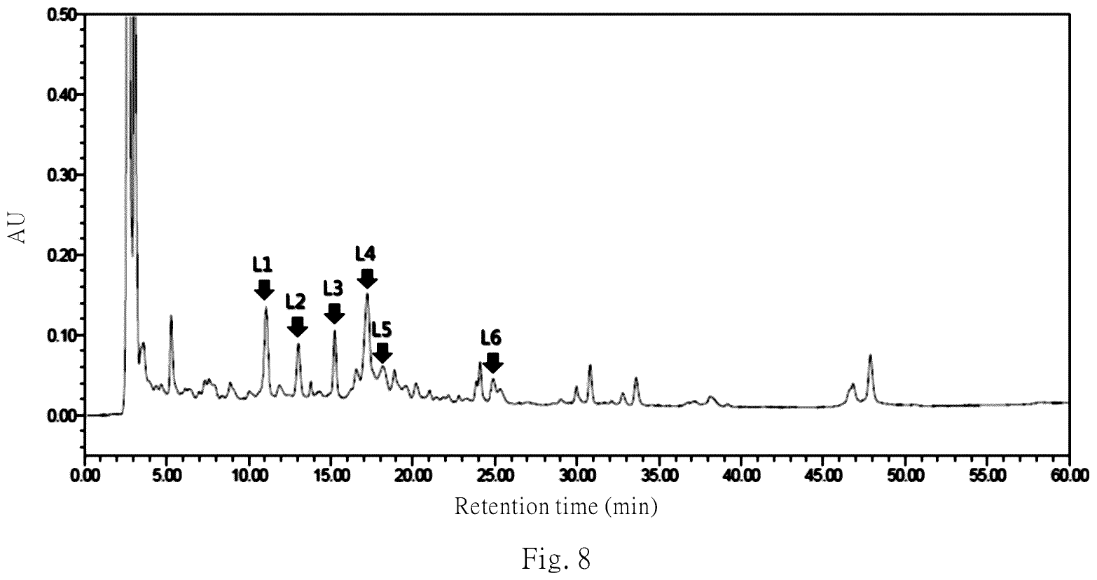

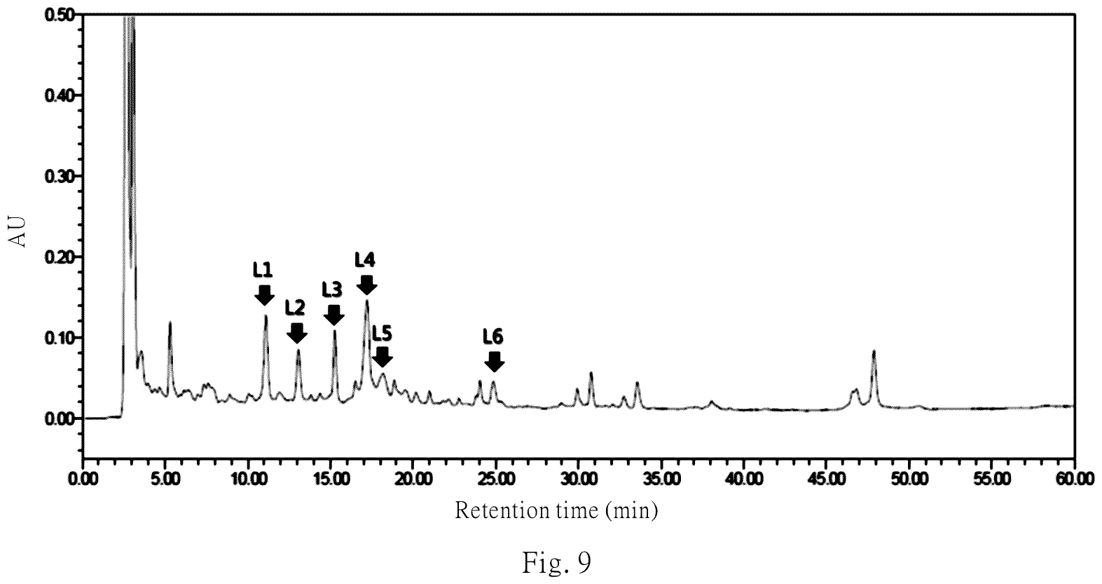

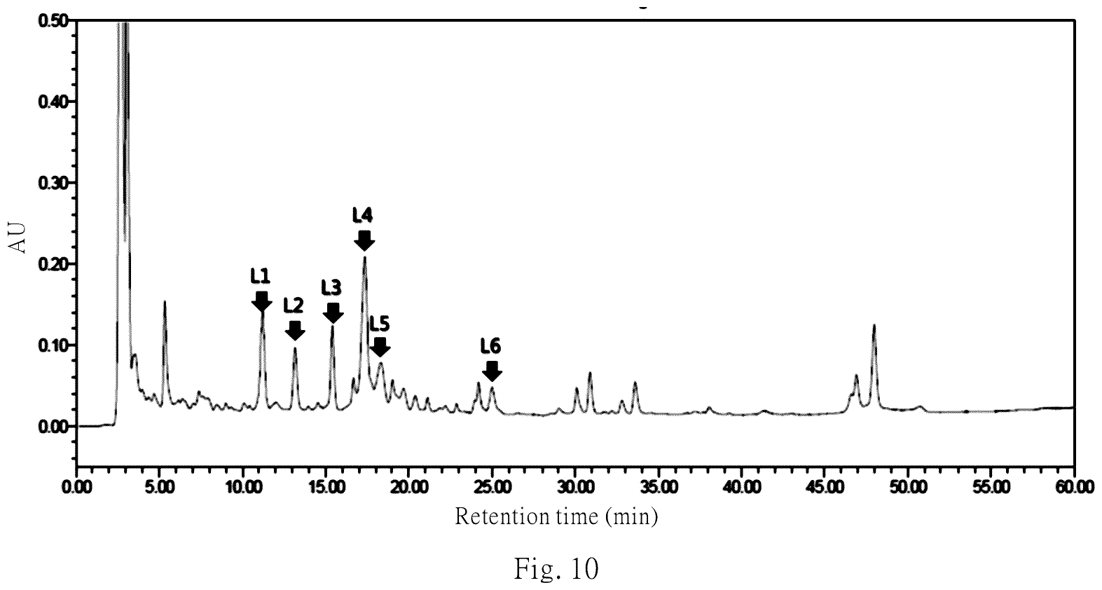

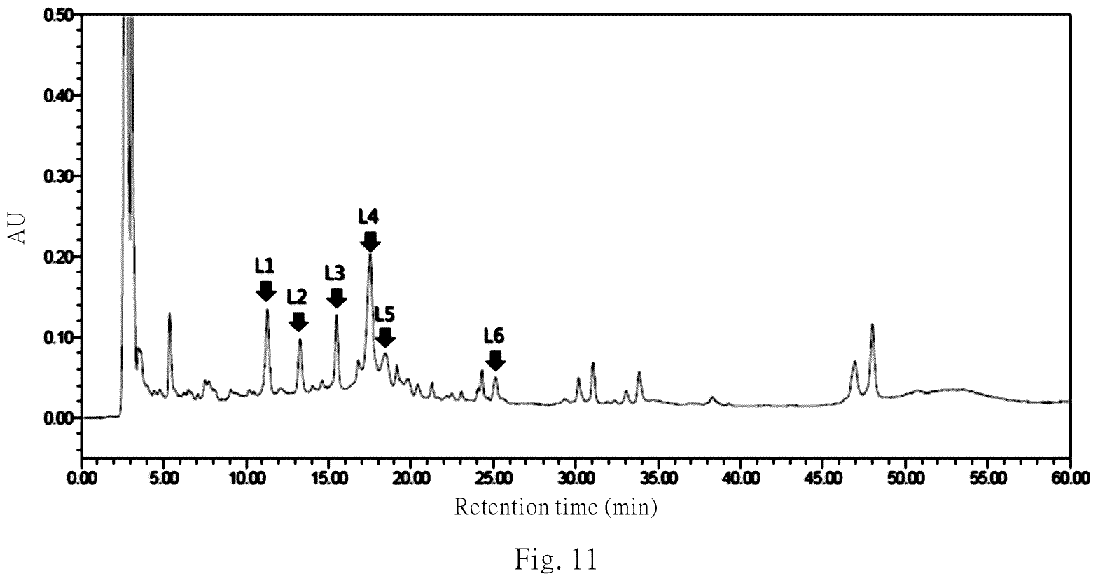

To demonstrate that the plant peptide group disclosed herein can be separated consistently, the second embodiment of the present invention involved sequentially performing a water-based extraction process and a supercritical extraction process on each of a variety of cucurbitaceous plants of different sources, namely pumpkin, watermelon, bitter melon, and wild bitter melon, in order to obtain an initial product of each of the foregoing plant-based raw materials. The initial product of each plant-based raw material was subjected to the aforesaid pre-treatment process and then to an HPLC analysis under the aforesaid analysis conditions. The resulting chromatograms are shown in FIG. 2 to FIG. 11. It should be pointed out that in BRIEF DESCRIPTION OF THE SEVERAL VIEWS OF THE DRAWINGS, the number following the name of a cucurbitaceous plant is used to distinguish between the different batches or sources of the same kind of plant. The amount of the substance corresponding to each of the foregoing sections of each chromatogram is shown in Table 2 below.

TABLE 2
Amounts of the substances corresponding to
sections (L1) to (L6) in FIG. 2 to FIG. 11

| Amount | Section | | | | | |
|---|---|---|---|---|---|---|
| (ppm) | L1 | L2 | L3 | L4 | L5 | L6 |
| 1 | 992.21 | 995.63 | 945.99 | 584.64 | 661.69 | 890.75 |
| 2 | 1344.12 | 1192.05 | 1488.25 | 990.76 | 1234.60 | 823.62 |
| 3 | 2085.21 | 1675.05 | 1577.28 | 1487.69 | 2047.22 | 1106.59 |
| 4 | 1827.91 | 1501.76 | 1310.04 | 1500.26 | 1855.20 | 1182.93 |
| 5 | 2182.67 | 1831.10 | 1816.86 | 2406.93 | 3511.78 | 1273.86 |
| 6 | 1701.57 | 1341.18 | 1492.64 | 1861.20 | 1480.20 | 798.40 |
| 7 | 1785.66 | 1340.65 | 1271.48 | 1581.15 | 1714.35 | 1460.34 |
| 8 | 1671.49 | 1269.72 | 1354.72 | 1722.42 | 1805.11 | 989.11 |
| 9 | 1103.81 | 1210.26 | 909.06 | 1151.22 | 858.40 | 792.10 |
| 10 | 1748.75 | 1349.73 | 1378.44 | 2116.25 | 2417.00 | 760.33 |
| Mean ± | 1644.34 ± | 1370.71 ± | 1354.48 ± | 1540.25 ± | 1758.56 ± | 1007.80 ± |
| SD | 368.83 | 230.76 | 259.83 | 509.08 | 772.63 | 227.07 |
| Greatest | 2182.67 | 1831.10 | 1816.86 | 2406.93 | 3511.78 | 1460.34 |
| value | | | | | | |
| Smallest | 992.21 | 995.63 | 909.06 | 584.64 | 661.69 | 760.33 |
| value | | | | | | |

It can be seen in the chromatograms in FIG. 2 to FIG. 11 that the occurrences of sections (L1) to (L6) correspond to a range of retention times from 10.5 to 25.5 minutes. More specifically, the retention time of section (L1) was 10.5-11.5 minutes, the retention time of section (L2) was 12.5-13.5 minutes, the retention time of section (L3) was 14.5-15.5 minutes, the retention time of section (L4) was 16.5-17.5 minute, the retention time of section (L5) was 17.5-18.5 minutes, and the retention time of section (L6) was 24.5-25.5 minutes.

It can be known from the chromatograms in FIG. 1 to FIG. 11 that the plant peptide group disclosed herein was consistently present in the initial product of each batch of plant-based raw material and in the initial products of each kind of plant-based raw material. This demonstrates the availability and reproducibility of the plant peptide group disclosed herein.

The third embodiment of the present invention discloses the same plant peptide group as in the previous embodiments, except that the pre-treatment process performed on the initial product of the plant-based raw material was different. The pre-treatment process for the third embodiment was carried out as follows:

The initial product of the plant-based raw material was dissolved in 30% acetonitrile and then went through the first ultrasonic extraction, with the extraction time being 20 minutes, the water bath temperature being lower than room temperature, and the centrifugation conditions being 9000 rmp for 5 minutes. After separation, a first supernatant and a first precipitate were obtained. The first precipitate was dissolved in 30% acetonitrile and then went through the second ultrasonic extraction, with the extraction time being 20 minutes, the water bath temperature being lower than room temperature, and the centrifugation conditions being 9000 rmp for 5 minutes. After separation, a second supernatant was obtained. The first supernatant and the second supernatant were collected and filtered through a membrane filter having a pore size of 0.20-0.25 μm to produce a filtrate for use in the HPLC analysis that followed.

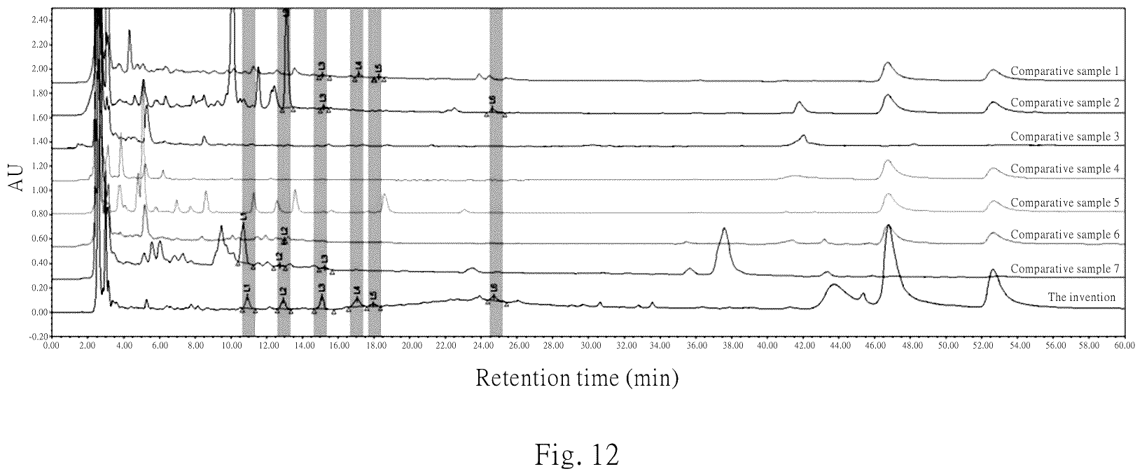

Besides, to prove the novelty and uniqueness of the plant peptide group disclosed herein, the third embodiment further involved performing the pre-treatment process in the first embodiment on commercially available bitter melon extracts (comparative samples 1-7), performing an HPLC analysis on each of the pre-treated comparative samples 1-7 under the same analysis conditions as in the first embodiment, and combining the resulting chromatograms of comparative samples 1-7 with the chromatogram in FIG. 1, as shown in FIG. 12.

It can be known from the results in FIG. 12 that, while the plant peptide group disclosed herein includes peptides corresponding to sections (L1) to (L6), comparative sample 1 lacked sections (L1), (L2), and (L6); comparative sample 2 lacked sections (L1), (L4), and (L5); comparative sample 3 lacked sections (L1) to (L6); comparative sample 4 lacked sections (L1) to (L6); comparative sample 5 lacked sections (L1) to (L6); comparative sample 6 lacked sections (L1), (L3), and (L6); and comparative sample 7 lacked sections (LA) to (L6). The results prove that the plant peptide group disclosed herein is novel and unique and cannot be obtained by separation from other extracts of the same source plant.

To prove that the plant peptide group disclosed herein can simultaneously treat or improve symptoms associated with blood pressure abnormality, blood glucose abnormality, and blood lipid abnormality, the following efficacy tests were conducted.

First, using the dosage of 300 mg/60 Kg adult body weight/day for humans as a reference, the dosage for rats was obtained by conversion based on general common knowledge of a person of ordinary skill in the art. The following tests were designed and conducted for different animal models, and all the test durations were eight weeks.

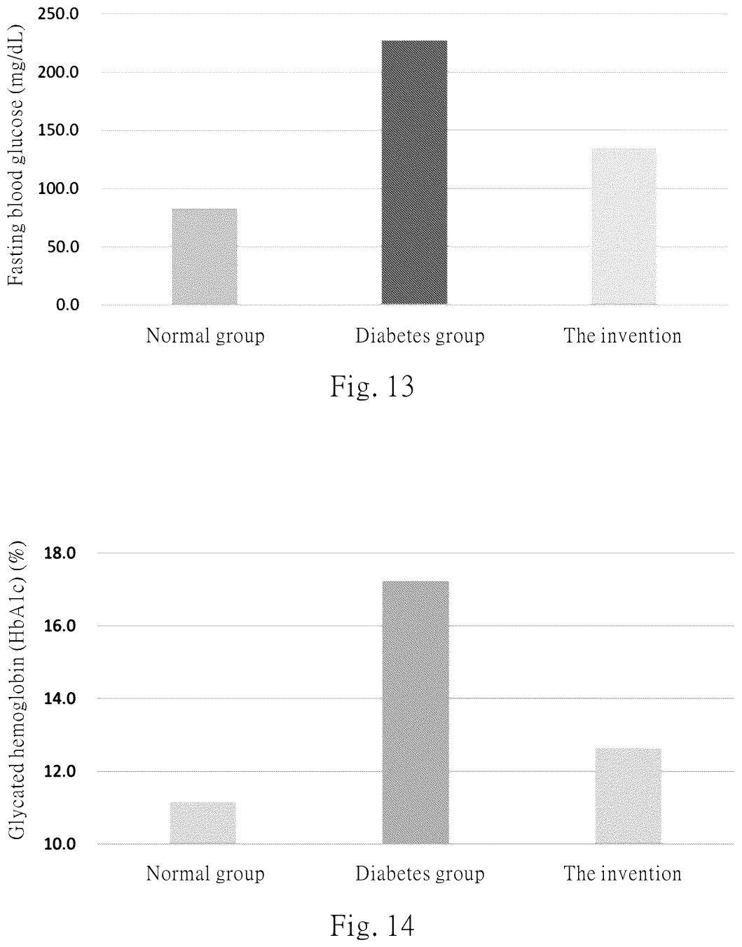

Animal test (1): SD rats were randomly divided into three groups. Group 1 was normal rats whereas group 2 and group 3 were rats of a type-2 diabetes model induced jointly by drug STZ (25 mg/kg body weight) and a high-fat diet. The plant peptide group disclosed herein was continuously administered to group 3 during the test. At the end of week 8, the fasting blood glucose (FBG) and glycated hemoglobin (HbAlc) of each group of rats were tested, and the results are shown in FIG. 13 and FIG. 14.

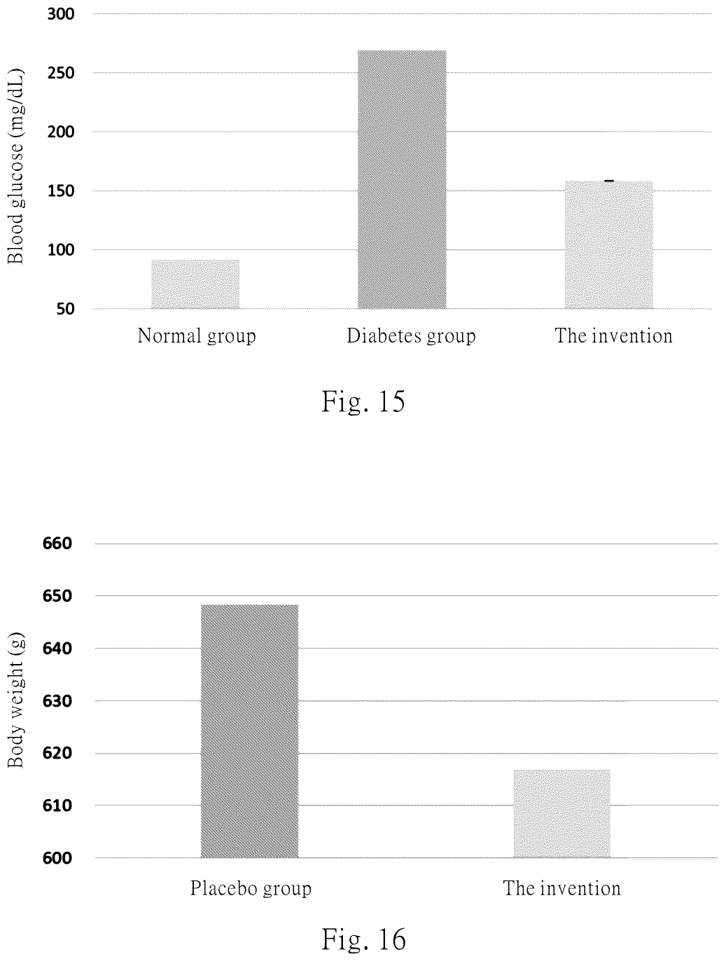

Animal test (2): SD rats were randomly divided into three groups. Group 1 was normal rats whereas group 2 and group 3 were rats of a type-1 diabetes model induced by drug STZ (75 mg/kg body weight). The plant peptide group disclosed herein was continuously administered to group 3 during the test. At the end of week 8, the fasting blood glucose of each group of rats was tested, and the results are shown in FIG. 15.

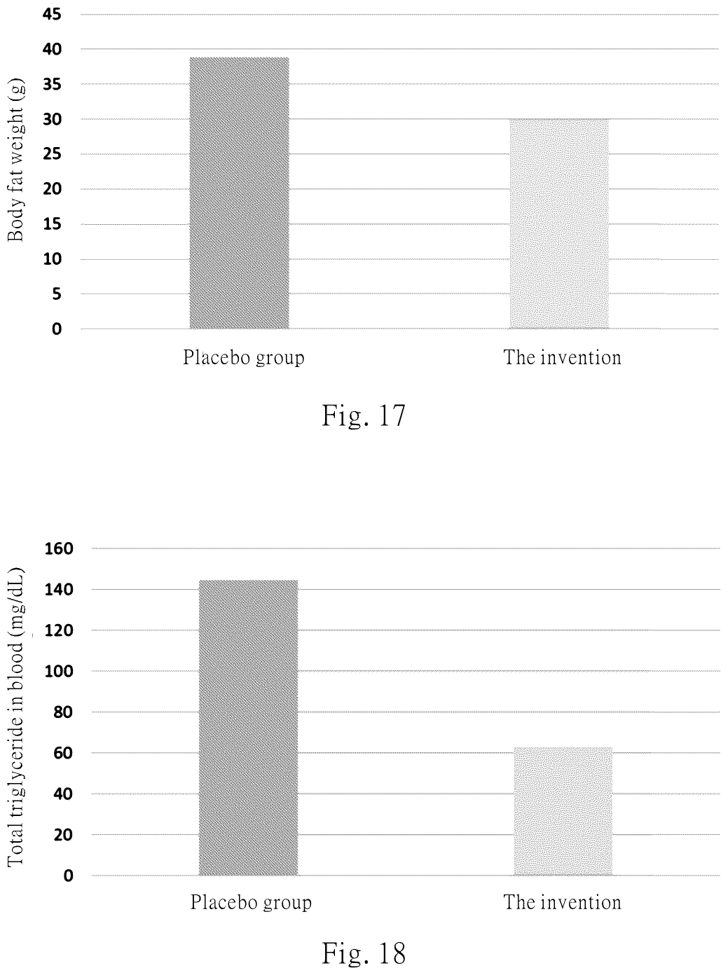

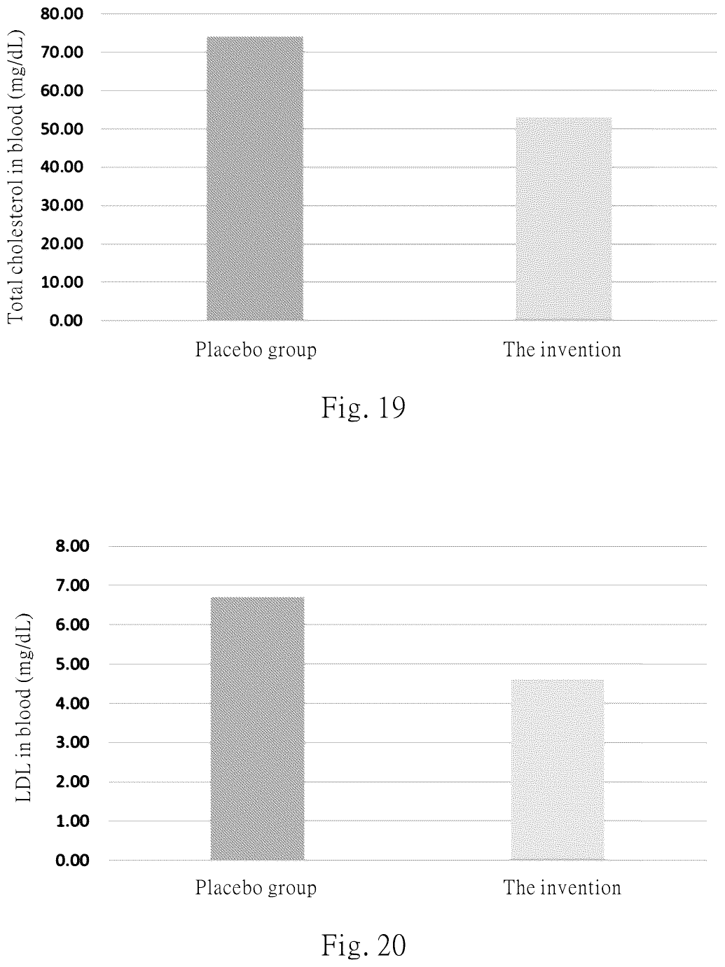

Animal test (3): SD rats were randomly divided into two groups. Both groups of rats were given a high-fat diet, and an eight-week test was conducted after the body weight of each group of rats had increased by 10%. During the test, the high-fat diet remained, but only group 2 of rats was fed with the plant peptide group disclosed herein. At the end of the test, the body weight, body fat, triglycerides in blood, total cholesterol in blood, and low-density cholesterol (or low-density lipoprotein (LDL) to be exact) in blood of each group of rats were tested, and the results are shown in FIG. 16 to FIG. 20.

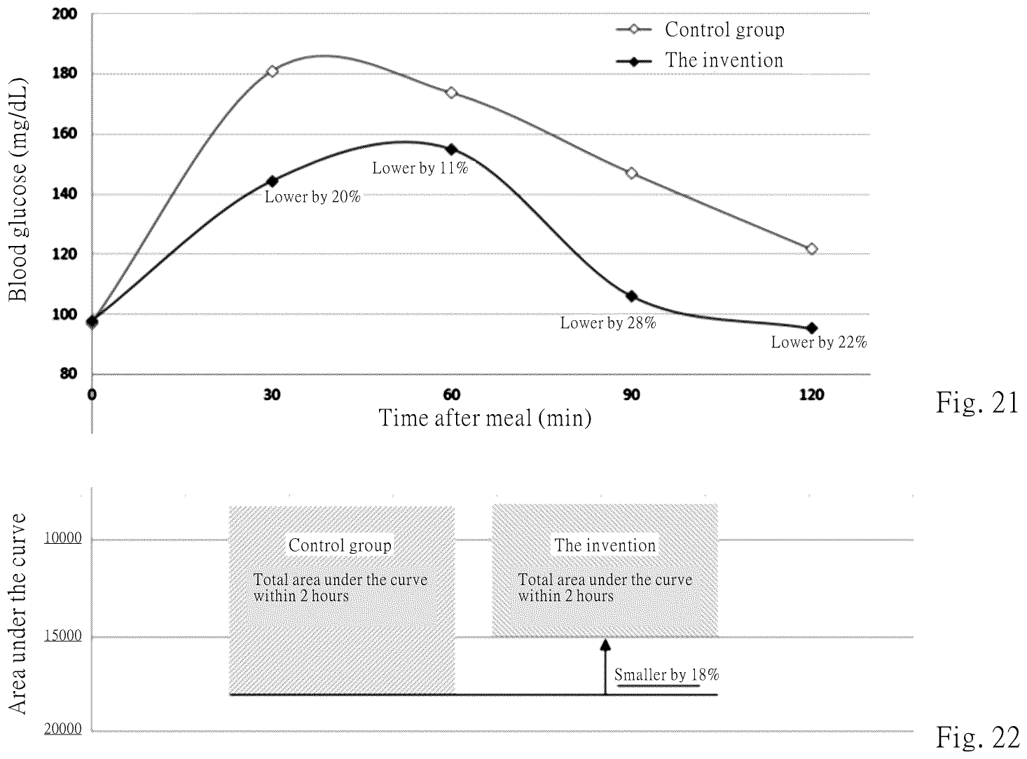

In addition, a plurality of test subjects were randomly divided into two groups. Group 1 was not given the plant peptide group disclosed herein, but group 2 was (at 300 mg/60 Kg adult body weight/day). Each group of test subjects drank 75 g of glucose while fasting and had their blood glucose value tested 0, 30, 60, 90, and 120 minutes after the glucose was drunken. After the 2 hours, the area under the curve of the oral glucose tolerance test (OGTT AUC) of each group was analyzed. The results are shown in FIG. 21 and FIG. 22.

The results of the animal tests and the human subject research are summarized in Table 3 below. It can be known from the results in Table 1 and FIG. 12 to FIG. 21 that the plant peptide group disclosed herein has an improving effect on both inherited and acquired blood glucose metabolic abnormalities and, even given a sustained high-fat diet, is still effective in reducing the effect of the high-fat diet on blood lipids and cholesterol. Therefore, it can be inferred that the plant peptide group disclosed herein is indeed capable of improving multiple abnormal metabolic indicators at the same time, of simultaneously improving symptoms associated with hyperglycemia, hyperlipidemia, and obesity, and of reducing the risk of having diseases caused by the aforesaid metabolic disorders.

TABLE 3
Efficacy analysis results

| | | | | Blood glucose, |
|---|---|---|---|---|
| | | | OGTT | two hours |
| Analysis item | FBG | HbA1c | AUC | after meal |
| As compared | −40.4% | −26.7% | −17.9% | −21.7% |
| with the | | | | |
| control group | | | | |

| | | | | Total | |
|---|---|---|---|---|---|
| | Body | Body | Triglycerides | cholesterol | LDL in |
| Analysis item | weight | fat | in blood | in blood | blood |
| As compared | −4.8% | −23.2% | −56.3% | −28.2% | −31.2% |
| with the | | | | | |
| control group | | | | | |

Furthermore, to show the difference in effect between the plant peptide group disclosed herein and compositions lacking the peptide group corresponding to sections (L1) to (L6), rats of a type-2 diabetes model were separately fed with the plant peptide group disclosed herein, comparative sample 1, and comparative sample 4, and the changes in body weight, FBG (fasting blood glucose), and HbA1C of each experimental group of rats were analyzed, as shown in Table 4. For the process flow of the experiment, please refer back to the description of animal test (1). The data in Table 4 is the ratios of change of each experimental group in comparison with the control group of rats of the same diabetes model.

TABLE 4
Ratios of change of each experimental group in comparison
with the control group of rats of the same diabetes model

| | Comparative | Comparative | The |
|---|---|---|---|
| | sample 1 | sample 4 | invention |
| Body weight | −1.4% | −3.9% | −4.8% |
| FBG | +13.8% | −2.2% | −40.4% |
| HbA1c | 5.3% | −9.3% | −26.7% |

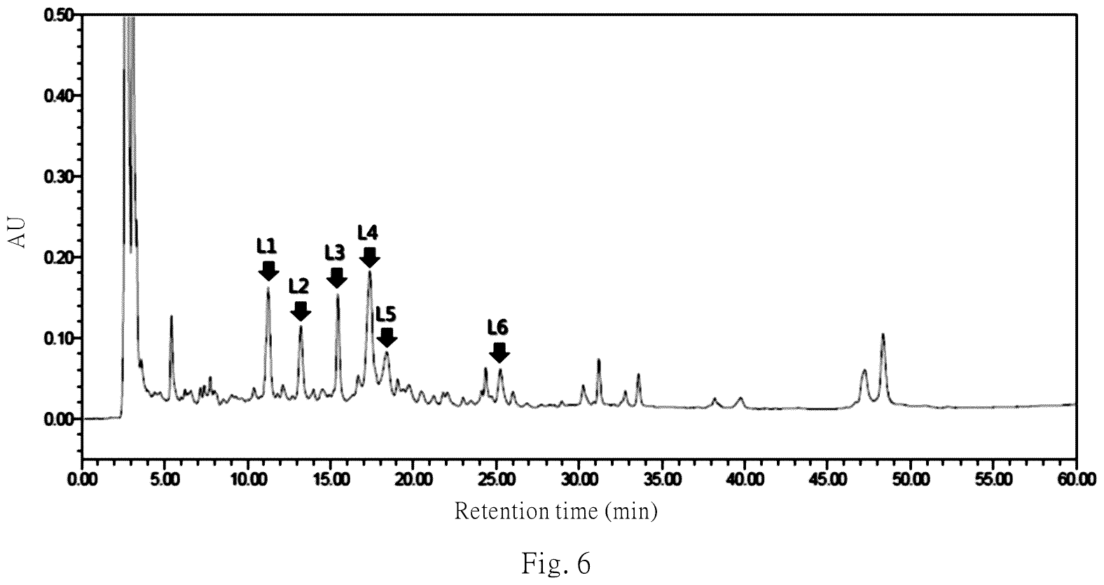

Moreover, rats of a high-fat diet model were separately fed with the plant peptide group disclosed herein and comparative sample 4, and the changes in body weight, body fat, triglycerides, and total cholesterol of each experimental group of rats were analyzed, as shown in FIG. 6. For the process flow of the experiment, please refer back to the description of animal test (3). The data in Table 5 is the ratios of change of each experimental group in comparison with the control group of rats of the same diabetes model.

TABLE 5
Ratios of change of each experimental group in comparison
with the control group of rats of the same diabetes model

| | Comparative | The |
|---|---|---|
| | sample 4 | invention |
| Body weight | −3.1% | −4.8% |
| Body fat | −9.1% | −23.2% |
| Triglycerides | −13.0% | −56.3% |
| Total cholesterol | −21.5% | −28.2% |

The results in Table 4 and Table 5 prove that the plant peptide group disclosed herein is significantly more effective in regulating blood glucose and blood lipids than compositions lacking the peptide group corresponding to sections (L1) to (L6).

In addition, to analyze the blood pressure reduction activity of the plant peptide group disclosed herein, the activity of angiotensin-converting enzyme (ACE) was tested by liquid chromatography (LC). More specifically, hippuryl-L-histidyl-L-leucine (hip-his-leu, or HHL) can be used as a substrate and reacted with ACE to produce hippuric acid (HA) and HL dipeptide (his-leu). If the activity of ACE is inhibited after a test sample is added, the amounts of the hippuric acid and HL dipeptide produced will be reduced, indicating that the test sample can treat or improve hypertension effectively. In this test, the amount of the hippuric acid produced after adding the plant peptide group disclosed herein was compared with that produced without adding the plant peptide group, in order to analyze whether or not the plant peptide group can lower blood pressure. The rate of inhibition of ACE activity was calculated by using HPLC to determine the amounts of the hippuric acid produced (areas of integral). It can be known from the test results that the test group, to which the plant peptide group disclosed herein was added (at 10 mg/mL), lowered blood pressure by 30.63% in comparison with the control group, which was not added with the plant peptide group disclosed herein. This shows that the plant peptide group disclosed herein is indeed capable of lowering blood pressure and can be used to prepare a composition for treating or improving hypertension and related symptoms.

1. A method for treating or preventing a metabolic disease with an isolated plant peptide group, comprising: administering an effective amount of the plant peptide group or of a composition containing the plant peptide group to an individual to bring the individual's blood lipids, blood pressure, and blood glucose back to normal levels;
wherein the metabolic disease has symptoms of a combination of at least two of hyperlipidemia, hypertension, and hyperglycemia;
wherein the plant peptide group is isolated from an initial product of a plant-based raw material, and the plant peptide group comprises peptides corresponding to sections (L1) to (L6) of a chromatogram of high-performance liquid chromatography (HPLC), with the sections (L1) to (L6) corresponding to molecular weights in a range from 700 Da to 2400 Da, the section (L1) having a retention time of 10.5-11.5 minutes, the section (L2) having a retention time of 12.5-13.5 minutes, the section (L3) having a retention time of 14.5-15.5 minutes, the section (L4) having a retention time of 16.5-17.5 minutes, the section (L5) having a retention time of 17.5-18.5 minutes, and the section (L6) having a retention time of 24.5-25.5 minutes;
wherein the chromatogram of HPLC is obtained by performing a pre-treatment process on the initial product of the plant-based raw material and performing an analysis with an HPLC instrument under predetermined analysis conditions;
wherein the analysis conditions comprise: a detection wavelength of 220 nm, an octadecyl carbon chain (C18)-bonded silica column serving as a separation column, a column temperature of 60° C., eluent A being 5% acetonitrile containing 0.10% trifluoroacetic acid, eluent B being 95% acetonitrile containing 0.085% trifluoroacetic acid, and a mobile phase including 10%-20% said eluent B used from a 0-minute time point through a 10-minute time point, 20%-30% said eluent B used from the 10-minute time point through a 25-minute time point, and 30%-60% said eluent B used from a 20-minute time point through a 60-minute time point.

2. The method for treating or preventing the metabolic disease with the isolated plant peptide group as claimed in claim 1, wherein the pre-treatment process comprises:
step a: dissolving the initial product of the plant-based raw material in 30% acetonitrile, and then performing ultrasonic extraction to obtain a supernatant; and
step b: filtering the supernatant to obtain a filtrate to be analyzed by HPLC.

3. The method for treating or preventing the metabolic disease with the isolated plant peptide group as claimed in claim 2, wherein the step b comprises filtering with a filter having a pore size in a range from 0.20 to 0.25 μm.

4. The method for treating or preventing the metabolic disease with the isolated plant peptide group as claimed in claim 1, wherein the plant peptide group has an effective dosage of 5 mg/kg/day for humans.

5. The method for treating or preventing the metabolic disease with the isolated plant peptide group as claimed in claim 1, wherein the metabolic disease is obesity.

6. The method for treating or preventing the metabolic disease with the isolated plant peptide group as claimed in claim 1, wherein the metabolic disease is hyperlipidemia.

7. The method for treating or preventing the metabolic disease with the isolated plant peptide group as claimed in claim 1, wherein the metabolic disease is diabetes.

8. The method for treating or preventing the metabolic disease with the isolated plant peptide group as claimed in claim 1, wherein the metabolic disease is kidney disease.

9. The method for treating or preventing the metabolic disease with the isolated plant peptide group as claimed in claim 1, wherein the plant peptide group is isolated from a cucurbitaceous plant.

10. The method for treating or preventing the metabolic disease with the isolated plant peptide group as claimed in claim 42, wherein the cucurbitaceous plant is bitter melon, wild bitter melon, pumpkin, or watermelon.
